(19) United States
(12) Patent Application Publication
(10) Pub. No.: US 2022/0015639 A1
(43) Pub. Date: Jan. 20, 2022

(54) 2D AND 3D IMAGING SYSTEM FOR A CUTANEOUS PIGMENT DISORDER

(71) Applicants: THALES, COURBEVOIE (FR); SISPIA, Vincennes (FR)

(72) Inventors: Ion BERECHET, Vincennes (FR); Gérard BERGINC, Elancourt (FR); Stefan BERECHET, Joinville le Pont (FR)

(21) Appl. No.: 17/291,598

(22) PCT Filed: Nov. 6, 2019
(86) PCT No.: PCT/EP2019/080330

§ 371 Date: May 5, 2021

(30) Foreign Application Priority Data
Nov. 15, 2018 (FR) 1871843

(51) Int. Cl. A61B 5/00; G06T 11/00

(52) U.S. Cl. CPC A61B 5/0077; G06T 11/006; A61B 5/6898; A61B 5/0073; A61B 5/443

(57) ABSTRACT

An imaging system for a cutaneous pigment disorder which includes a device for acquiring 2D images of the pigment disorder, comprising: a light source comprising at least one emitter and receivers, a unit for processing the 2D images acquired, means for displaying the images processed. The invention further comprises: means for positioning the receivers at at least two viewing angles, which positioning means are distributed along a spherical cap, a structure for protecting the acquisition device and an operator, comprising a receiver-positioning opening, the protection structure being intended to be positioned on the skin. The processing unit comprises a 3D reconstruction module with a reflective Radon transform in order to obtain a 3D image which is reconstructed from the 2D images processed and the display means further comprise means for displaying the reconstructed 3D image.

The field of the invention is that of imaging of pigment disorders of the skin.

Dermatology faces growing demand for increasingly early treatment of cutaneous pathologies. For the issue of pigment disorders of the skin, statistics from the World Health Organization (WHO) show:

2 to 3 million carcinomas, 132 000 cases of melanoma worldwide in 2011,

a doubling of the number of cases every 10 years since 1945,

in 15 to 20% of cases, the melanoma develops from a mole,

15% chance of survival at 5 years if detected late,

95% chance of recovery if detected early.

To diagnose pigment disorders of the skin (moles, benign lesions, malignant lesions, carcinomas, melanomas, etc.), the dermatological techniques are based on criteria called ABCDE. Thus, the evolution of a pigment disorder into cancer is characterized by:

asymmetry (A) of the melanoma,

irregular border (B),

unusual color (C),

changing diameter (D)

unusual evolution (E).

In addition, dermatologists attempt to assess the non-uniformity of the thickness of the pigment disorder using two-dimensional imaging techniques such as dermoscopy or dermatoscopy which are not suitable for reliable characterization of the evolution of the pigment disorder at depth. In some cases, to confirm the diagnosis, the expensive practice of biopsies and histopathology examinations is carried out.

The challenge for dermatologists is to identify suspicious “moles” as early as possible and to develop new semiotics, i.e. the correspondence between what is observed and the pathology.

In the context of the diagnosis of pigment disorders, the most common technique, epiluminescence or dermatoscopy, is based on the use of a video microscope, most often digital, so that the image can be analyzed using the well-known ABCD criteria. More recently, new equipment has appeared, such as, for example, the Siascope from Astron Clinica and the MelaFind from Electro-Optical Systems. They are both based on multispectral information in the visible and near-infrared and differ in the associated information processing tools.

The Siascope is associated with a simple physical model and provides the user with calculated images of the melanin and hemoglobin. In a more advanced version, the practitioner has information on the localization of the melanin at depth, which is very important information for the diagnosis of certain pigmented lesions. However, the localization at depth is linked to the accuracy of the mathematical model used. A complete model of backscattering of light from the skin and its internal structure remains very difficult to obtain, very little data being available in the literature to validate this approach.

The MelaFind system, as the name suggests, analyzes images of pigment spots for the sole purpose of detecting melanoma. The images of these pigment spots are orthoimages. The associated digital tools are not based on a physical model, but essentially consist of a classification algorithm which compares any new spot with the existing database and returns a response of yes/no type to the operator. This type of device may be up to the task of diagnosis, or screening or prevention, but not to the task of therapeutic action and follow-up.

In practice, a skin biopsy with histology is used in addition. However, this approach is ill-suited to considering the subject's biochemical parameters. It remains dominated by Breslow's index, measuring the maximum thickness of the tumor on a histological section, and is therefore limited by the number and the quality of the sections, and is artificially reduced by regressive aspects with standardized consensual but empirical margins dependent on the thickness of the tumor as assessed by Breslow; the margins are not always compatible with the topography of the tumor (face), they are standardized and dependent on the clinically visible size of the tumor (to 1 cm) and are difficult to specify in certain clinical forms (morpheaform and micronodular basal-cell carcinoma).

There are many other cutaneous pathologies of various origins but they are often linked to the main chromophores of the skin, with devices dedicated to a single pathology (as in the case of MelaFind, as described above).

The aim of the invention is to alleviate these drawbacks.

More specifically, the subject of the invention is an imaging system for a cutaneous pigment disorder which comprises:

a device for acquiring 2D images of the pigment disorder comprising:
a light source configured to illuminate said disorder comprising
at least one emitter and
receivers,
a unit for processing the acquired images,
means for visualizing the processed images.

It is primarily characterized in that it further comprises:
means for positioning the receivers at at least two viewing angles so as to obtain at least one image per viewing angle, which means are distributed over a spherical cap,
a structure for protecting the acquisition device and an operator, comprising a window for positioning the receivers so as to direct them toward the pigment disorder, the structure being intended to be positioned on the skin and having an external surface that is opacified except for over the positioning window,
in that the processing unit comprises a module for 3D reconstruction using reflection Radon transform so as to obtain a 3D image reconstructed on the basis of the processed 2D images,
and in that the visualization means further comprise means for visualizing the reconstructed 3D image.

The dimensions of the protective structure are advantageously compatible with a portable acquisition device.

According to one feature of the invention, the acquisition device comprises a multicellular structure with one receiver per cell.

According to another feature of the invention, the acquisition device comprises a light source emitting in the visible band, and means for acquiring the images simultaneously via the receivers.

The positioning means comprise, for example, a rail on which one or more receivers are positioned, and means for rotating the rail. The rail may be a sliding rail with a single receiver or a single emitter-receiver.

The light source may be multiwavelength in the visible and near-infrared bands, and/or the band of the first therapeutic window and/or the band of the second therapeutic window and/or the SWIR band, the one or more receivers corresponding to said wavelengths and being synchronized with the emitters, and the acquisition device comprising means for acquiring the images successively via the receivers at a rate of at least one image per wavelength and per viewing angle.

By virtue in particular of the illumination of the skin in the visible and near-infrared bands (0.4 μm to 1.1 μm) and/or SWIR (short-wave infrared, from 1.1 μm to 2.2 μm), and/or those of the first or second therapeutic window, this novel imaging system allows 2D visualization at various viewing angles, 3D volume visualization of pigment disorders and their cutaneous roots, using a non-invasive optical device and associated 2D and 3D visualization and processing modules.

The system, which is based on volume illumination, allows direct observation after three-dimensional reconstruction of the pigment disorder through its depth.

The system according to the invention is sufficiently generic to be adapted to, if not all, at least a large number of skin pathologies. By virtue of the 3D volume visualization which allows both external and internal viewing of the pigment disorder, it makes it possible to reveal structures and signs that are invisible to the naked eye (internal), to improve the performance of the clinical diagnosis of pigmented lesions, to detect parameters allowing the differential diagnosis between melanoma and nevus to be to refined (localization, quantity or quality of melanin, highlighting of neovascularization around the melanoma, depth of the melanoma structure, etc.).

It is possible, by virtue of the 3D volume imaging combined with the 2D two-dimensional imaging at various viewing angles, to better view the three-dimensional limits of a pigment disorder and of a potential tumor, to be certain of the complete exeresis of a malign cutaneous tumor, the conditions for healing and improve the functional prognosis.

Other features and advantages of the invention will become apparent from reading the following detailed description, given by way of non-limiting example and with reference to the appended drawings, in which:

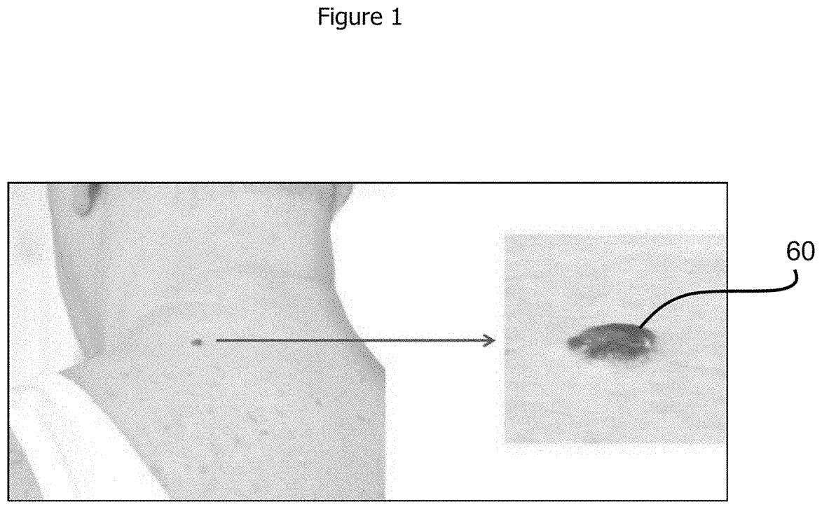

FIG. 1 is an illustration of a pigment disorder, for example a nevus,

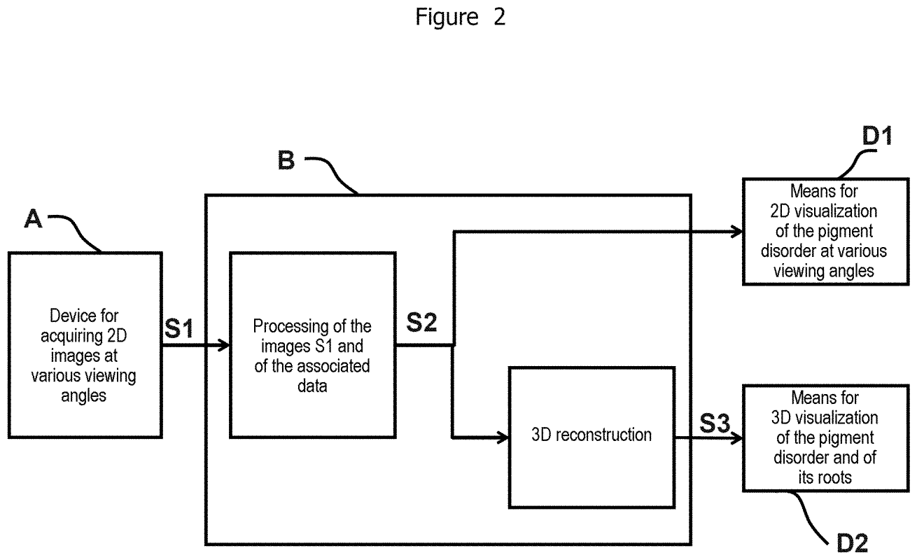

FIG. 2 schematically shows an exemplary imaging system according to the invention,

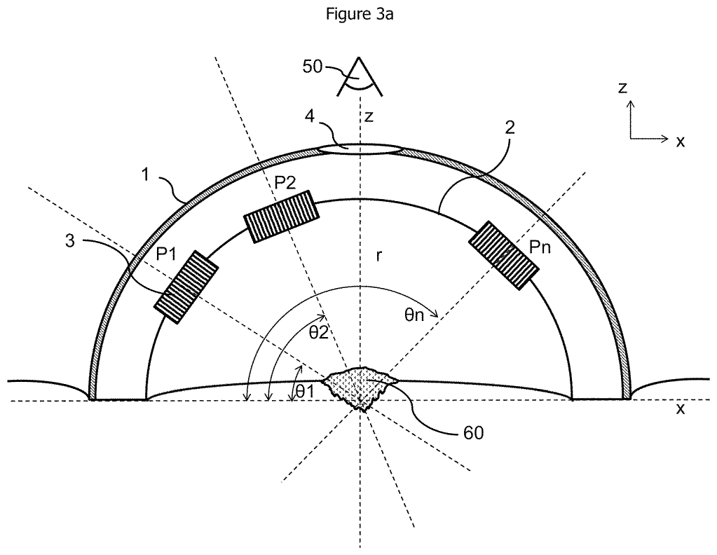

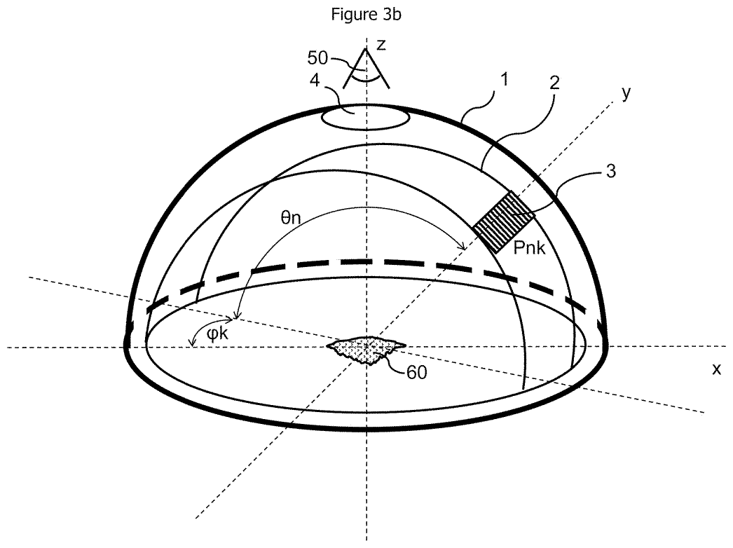

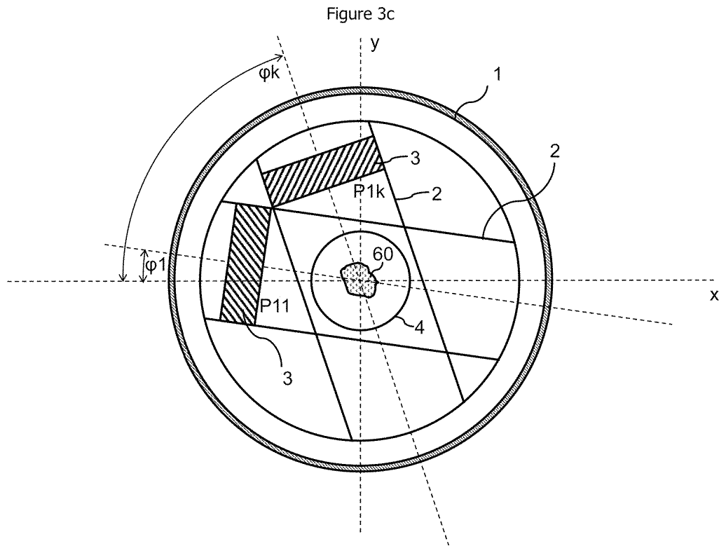

FIG. 3a shows a vertical cross section of a device for acquiring 2D images at various angles in a dome version,

FIG. 3b shows a perspective view of a device for acquiring 2D images at various angles in a dome version,

FIG. 3c shows a horizontal cross section of a device for acquiring 2D images at various angles in a dome version,

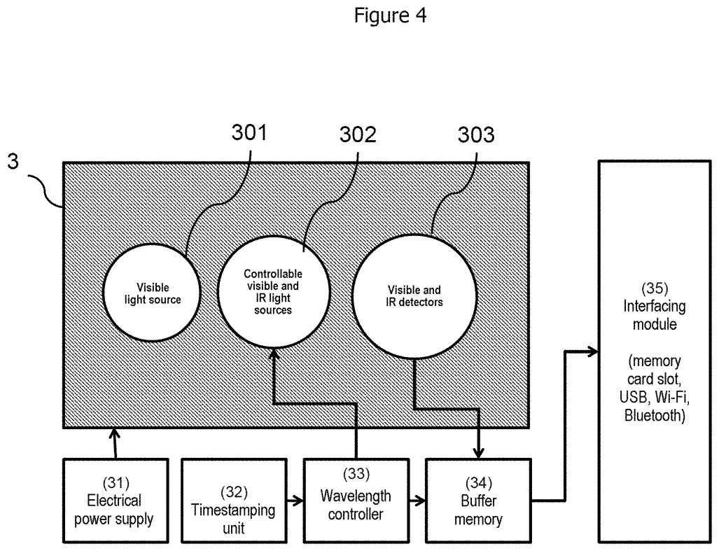

FIG. 4 schematically shows the functional elements of a device for acquiring 2D images,

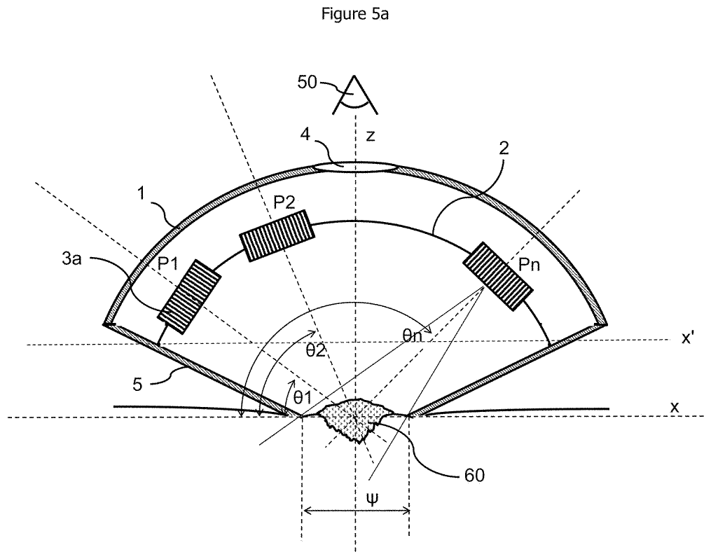

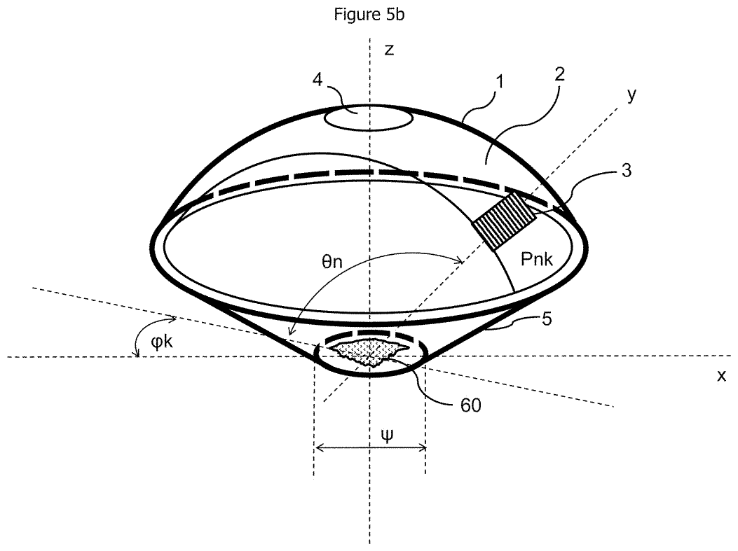

FIG. 5a shows a vertical cross section of a device for acquiring 2D images at various angles in a dome version with a skirt,

FIG. 5b shows a perspective view of a device for acquiring 2D images at various angles in a dome version with a skirt,

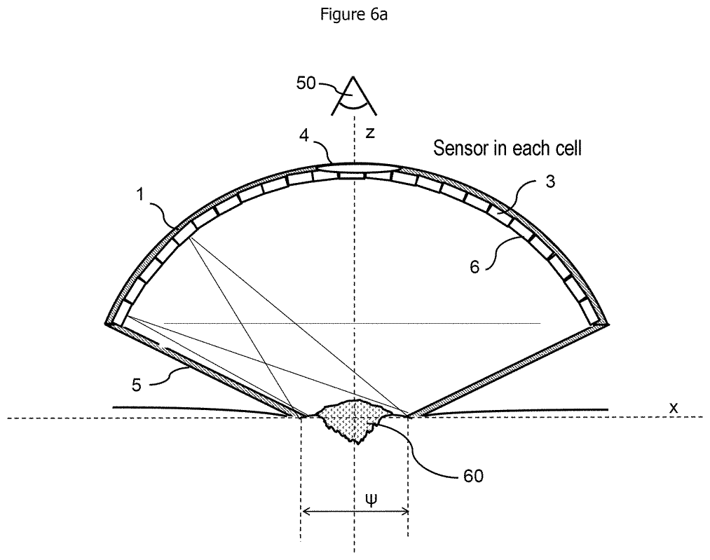

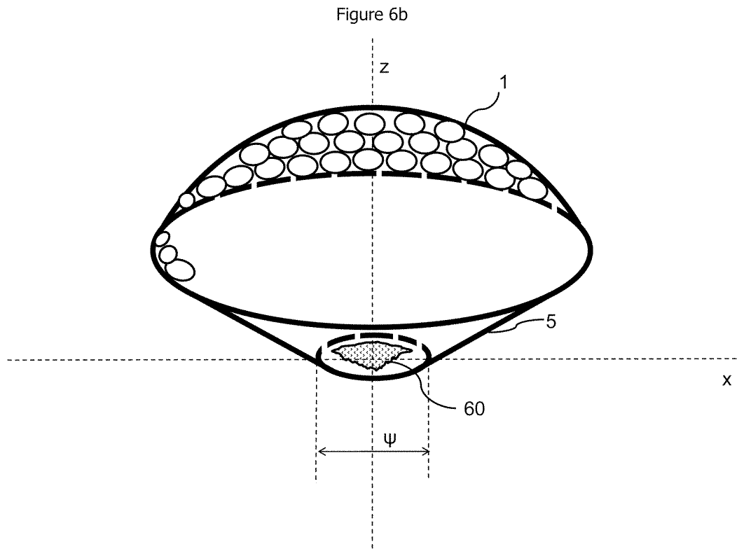

FIG. 6a shows a vertical cross section of a device for acquiring 2D images at various angles in a multicellular dome version with a skirt,

FIG. 6b shows a perspective view of a device for acquiring 2D images at various angles in a multicellular dome version with a skirt,

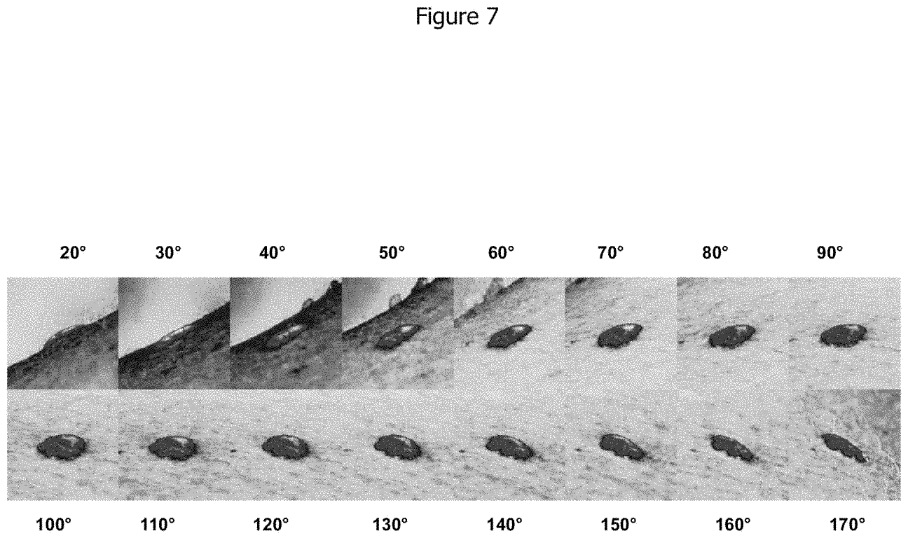

FIG. 7 is an illustration of 2D images of a nevus acquired at various angles and post-processed,

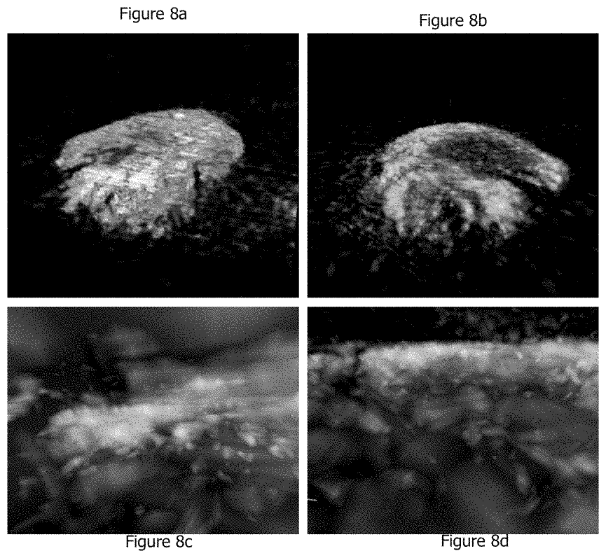

FIG. 8 form a set of illustrations of the three-dimensional reconstruction, by rendering voxels, of the complete 3D volume of a nevus and of close-ups of the three-dimensional structure of the nevus in question, with, for FIG. 8a, an image of the surface of the nevus at one viewing angle, for FIG. 8b, an image of the surface of the nevus from another viewing angle, for FIG. 8c, a depth image of the nevus from one viewing angle, for FIG. 8d, a depth image of the nevus from another viewing angle.

From one figure to another, the same elements bear the same references.

According to the invention, the imaging system for pigment disorders described with reference to FIG. 2 is intended to be used by an operator, who may be a healthcare professional but not necessarily so. The imaging system comprises the following elements:

A device A for acquiring 2D images at various viewing angles and potentially at various wavelengths for each viewing angle, which generates raw 2D images S1 and the associated data (viewing angles and wavelength).

An image processing unit B which generates processed 2D images S2 from the raw images S1 and the associated data and which preferably also generates a 3D reconstruction S3 of the pigment disorder and of its roots on the basis of the processed images S2.

Means D1 for 2D visualization, at various viewing angles, of the pigment disorder on the basis of the processed 2D images S2.

Means D2 for 3D visualization of the pigment disorder and of its reconstructed roots S3.

The image processing unit B and/or the visualization means D1 and D2 may be remote from the acquisition device A.

The image processing unit B performs RAW conversions to an usable image format (JPEG, PNG, TIFF, etc.), corrections for optical aberrations, framing, centering, registration, calibration, scaling, thresholding, and angular indexing of the raw images S1. Processed images S2 are obtained. Examples of processed 2D images S2 of a nevus at various viewing angles (20°, 30°, . . . , 160°, 170°) are given in FIG. 7.

Preferably, as shown in FIG. 2, on the basis of the processed images S2 (themselves resulting from the images S1 representing the intensity levels of electromagnetic radiation reflected or emitted by the surface of the object), the unit B also provides the three-dimensional reconstruction of the pigment disorder via an algorithmic process of reflection tomography type based on the inverse Radon transform. Processed images S3 are obtained. Regarding this three-dimensional reconstruction, reference may be made, for example, to U.S. Pat. No. 8,836,762, “Optronic system and method dedicated to identification for formulating three-dimensional Images”. This type of reconstruction uses data linked to the electromagnetic emission, reflection or scattering of the light wave incident on the structures of the pigment disorder (nevus, carcinoma, melanoma, etc.) through its depth; it allows complete three-dimensional reconstruction (of tomographic type) of the pigment disorder as illustrated in FIG. 8, without introducing tissue absorption parameters.

These images S3 are of point-cloud, isodensity, voxel-rendering type (for example maximum intensity projection (MIP), as described in patent EP 3 234 914 B1: Method for discrimination and identification of objects of a scene by 3D imaging. The MIP technique makes it possible to visualize three-dimensional data in a two-dimensional plane. The voxels (volume pixels) are projected onto a 2D plane; the voxels are determined by the rays meeting the projection plane at the observation point and applying an imposed intensity threshold to the voxels. A plurality of projection planes are created at successive observation angles in order to obtain an impression of depth and thus improve the 3D rendering. An illustration of a rendering of the voxels of the reconstructed 3D volume (S3) of a nevus is given in FIG. 8: FIGS. 8a and 8b show an image of the surface of a nevus, taken at two viewing angles, FIGS. 8c and 8d show a depth image of the nevus taken at two viewing angles.

The 2D (images S2) and 3D (images S3) visualization means D1 and D2 are typically PCs, tablets, mobile telephones (“smartphones”) or any other visualization means.

The device A for acquiring 2D images, at various viewing angles, of the pigment disorder, shown in FIGS. 3a, 3b and 3c, will now be described in more detail. It comprises:

a light source configured to illuminate the pigment disorder. It comprises one or more emitters oriented toward the pigment disorder 60 (an example of which is shown in FIG. 1). The light source may be a visible source which emits in the 0.6 μm-0.8 μm band. Preferably, the light source is a single-wavelength and wavelength-controllable source and it emits successively in the visible and near-infrared bands (0.4 μm-1.1 μm), the bands of the first (0.65 μm-0.95 μm) and/or second (1 μm-1.35 μm) therapeutic windows and/or the SWIR band (1.1 μm-2.2 μm), with one wavelength per band.

one or more receivers 303 (shown in FIG. 4) matched to the wavelengths of the one or more emitters. The images S1 are obtained at the output of the receivers.

It is possible to have devices referred to as emitter-receivers 3 integrating the emitting function and the receiving function within the same device as shown in FIGS. 3a, 3b, 3c, 5a, 5b, 6a. An exemplary emitter-receiver 3 is detailed in FIG. 4. It comprises a light source 301 in the visible band, and also preferably light sources 302 in the visible and IR bands which are controllable, connected to a wavelength controller 33 which is itself connected to a timestamping unit 32. The wavelength controller 33 and the timestamping unit 32 may be hosted by the processing unit B. The device 3 also comprises receivers in the bands corresponding to those of the light sources (visible and IR).

means for positioning the one or more receivers (or even also the one or more emitters) at M viewing angles (M≥2) with respect to the pigment disorder which are distributed over a spherical cap as can be seen in FIGS. 3a, 3b, 5a and 5b, so as to obtain at least one image per viewing angle θn (n varies from 1 to N, with N≤M), the images being acquired at the same distance from the pigment disorder. The larger θN−θ1, the more precise the 2D and/or 3D visualized images will be. Typically, 120°≤θN−θ1≤180°; there are 16 viewing angles in FIG. 7 with θ1=20°, θN=170° and θN−θ1=150°. The positioning means comprise, for example, a rail 2 that is curved along this spherical cap, on which a receiver (or even an emitter-receiver 3) may slide to positions Pn ensuring a viewing angle θn. It is also possible to use a rail 2 to which receivers are attached at positions Pn determined beforehand (there are therefore N receivers), without having to slide them. The positioning means also comprise means for rotating the rail 2 about its vertical axis Oz (as shown in FIG. 3c in which two positions of the rail 2 can be seen) at angles φk (k varies from 1 to K, K≤M and N·K=M) ensuring positions Pnk of the one or more receivers (or of the emitter-receivers 3) as shown in FIGS. 3b and 3c or about a horizontal axis Ox located in a plane containing the ends of the rail. The larger φK−φ1, the more precise the 2D and/or 3D visualized images will be. Typically, 120°≤φK−φ1≤180°. According to one alternative, the positioning means are integral with the protective structure 1 described in greater detail below and it is the structure 1 which rotates manually or automatically. It is noted that θN−θ1=φK−φ1=180° corresponds to a dome-shaped structure as shown in FIGS. 3a and 3b.

According to one embodiment, the acquisition device comprises a multicellular structure, each cell 6 containing a receiver, or an emitter-receiver 3. This structure takes the shape of the spherical cap as shown in FIGS. 6a and 6b; it may be distributed over a limited area or over the entire inner surface of the cap.

The acquisition device may be adapted from or integrated into a mobile telephone (smartphone).

the acquisition device further comprises a structure 1 for protecting the operator 50 and the emitters and receivers, which generally follows the shape of the positioning means as shown in the figures, but other shapes may be envisaged (cubic, or compound or other). This structure 1 comprises a window for positioning the acquisition device so as to direct it toward the disorder 60. The positioning window is arranged at the apex of the protective structure 1, on the normal to the disorder 60 along the axis z (the skin is in a plane xy). Thus, the operator may position, by way of direct visual inspection, the acquisition device so that the window faces the disorder 60, as illustrated in FIGS. 3a and 3b, while keeping the protective structure 1 on the skin. This window may comprise a magnifying glass 4 facilitating visual centering on the pigment disorder 60 by the operator 50. The structure 1 is intended to be positioned on the skin by the operator and has an external surface that is opacified except for over the positioning window so as to protect the operator from light emissions and also to protect the receivers from stray emissions. For eye safety, the magnifying glass 4 advantageously comprises an automatic shutter during emission-reception.

FIGS. 3a and 3b show a structure 1 in the shape of a dome. According to one variant shown in FIGS. 5a and 5b, a skirt 5 is associated with the protective structure 1 for bearing against the skin and openings ψ that can be adjusted according to the dimensions of the pigment disorder 60 and/or to its position on this or that part of the body, such as the face for example.

Preferably, the dimensions of the structure 1 are compatible with a portable acquisition device A which is easy to place on various parts of the body: when the structure 1 forms for example a dome as shown in the figures, it typically has a radius of <15 cm. The structure may also be fixed and of larger dimensions.

By way of non-limiting example, the interfacing (35) is of the type: memory card slot, USB, Wi-Fi, Bluetooth, etc.

Two image acquisition techniques may be used:
“Passive” imaging technique: it concerns the visible and near-infrared bands. The pigment disorder is illuminated by the ambient light from outside the device passing through the positioning window, potentially equipped with the magnifying glass 4, which remains transparent to the ambient light. The emitter in question is then the ambient light. The 2D images are acquired successively for the device described with reference to FIGS. 5a and 5b. The images may be acquired simultaneously by the entire multicellular receiver structure for the acquisition device described with reference to FIGS. 6a and 6b. The acquisition device then comprises means for simultaneous acquisition, such as means for synchronizing the receivers. Ambient illumination makes it possible to obtain a 3D image of the surface volume of the disorder but does not always make it possible to obtain significant depth for the reconstructed three-dimensional image.

“Active” imaging technique: it concerns the visible and infrared bands. The pigment disorder is illuminated by the emitting sources 301, 302 of the emitter-receivers 3, the positioning window no longer being transparent to the ambient light from outside the device; this window is for example masked by the operator or may also be equipped with a shutter as indicated above. Illumination may be performed successively in a plurality of wavelengths for each viewing angle. The pigment disorder is then imaged successively for each wavelength and for each viewing angle. The acquisition device then comprises means for successive acquisition which are synchronized with the illuminations, such as means for synchronizing the receivers with the emitters. This technique makes it possible to obtain a 3D image of the surface volume of the disorder and a deep three-dimensional imaging of the pigment disorder. Advantageously, for each image capture, the emitting angle and the receiving angle are oriented and equal, or near-equal, with respect to the normal to the plane containing the skin. These angles (θ, φ) are illustrated in FIGS. 3a, 3b and 3c in the coordinate system (x, y, z). An angular coordinate system is thus defined with respect to the normal, where two angles on either side of the normal may have the same absolute value but are of opposite signs. Thus, the two-dimensional images are collected in a monostatic or near-monostatic configuration. The device is thus designed to use two-dimensional images with retroreflection or monostatic or near-monostatic reflection in order to obtain a three-dimensional image on the basis of all of the two-dimensional images taken at different angles. In addition, the precision of the 3D imaging will increase as the number of two-dimensional images collected and accurately referenced increases.

The invention makes it possible to obtain a three-dimensional volume image by way of illumination in the “first therapeutic window” (650 nm-950 nm); this approach results in increased spatial resolution and minimized background noise The use of three-dimensional imaging, the spectroscopic properties (photon absorption and emission) of which lie within the first therapeutic window, allows access to the imaging of “thick” tissues.

The “second therapeutic window” (1000 nm-1350 nm) makes it possible to increase the depth of penetration of the wave due to the minimization of photon scattering, and it is thus possible to obtain light transmission over several millimeters in depth. This emission window provides additional information on the evolution of the roots of the nevus and its microvascularization.

The scattering of the light wave from the various wavelengths used allows an array of three-dimensional reconstructions of pigment disorders and therefore complete and comparative information on pigment disorders given that the effects of scattering of light waves from pigment disorders are complementary (depth of scattering, scattering cross section, determination of the roughness of the scattering structure) according to the illumination wavelengths used.

Thus, the obtained two-dimensional images are used to obtain a three-dimensional reconstruction of the skin at depth, in particular for those illumination wavelengths having a high penetrating power with respect the tissues of the skin.

This 2D and 3D imaging system applies mainly to the biomedical field, to the identification of cutaneous or subcutaneous disorders. In the context of non-melanoma skin cancers (basal-cell carcinoma, squamous-cell carcinoma for example), the system according to the invention allows precise visualization of the three-dimensional limits of the tumor, which makes it possible to be certain of the complete exeresis of a malignant skin tumor which is a condition for healing.

Thus, the most frequent simple nodular forms (45 to 60% of cases) are very clearly delimited, but within these simple forms there may be a micronodular form, without peripheral delimitation and requiring greater margins of exeresis, the three-dimensional image allowing precise representation of these complex shapes.

Thus, the three-dimensional imaging makes it possible to reveal structures and signs that are invisible to the naked eye, improving the performance of the clinical diagnosis of pigmented lesions, to detect three-dimensional parameters allowing the differential diagnosis between melanomas, carcinomas and nevi to be refined (localization, shape, depth, highlighting of vascularization around the melanoma/carcinoma, changes to microrelief, changes to the dermoepidermal junction, vascular changes, visualization of the limits of the tumor which makes it possible to be certain of the complete exeresis of a skin tumor).

1. An imaging system for a cutaneous pigment disorder which comprises:
a device (A) for acquiring 2D images of the pigment disorder comprising:
a light source configured to illuminate said disorder comprising at least one emitter and
receivers,
a unit (B) for processing the acquired 2D images (S1), in order to obtain processed 2D images (S2),
means (D1) for visualizing the processed images (S2), further comprising:
means for positioning the receivers at at least two viewing angles so as to obtain at least one image per viewing angle, which means are distributed over a spherical cap,
a structure for protecting the acquisition device (A) and an operator, comprising a window for positioning the receivers so as to direct them toward the pigment disorder, the protective structure being intended to be positioned on the skin and having an external surface that is opacified except for over the positioning window,
in that the processing unit (B) comprises a module for 3D reconstruction using reflection Radon transform so as to obtain a 3D image (S3) reconstructed on the basis of the processed 2D images (S2),
and in that the visualization means further comprise means (D2) for visualizing the reconstructed 3D image (S3).

2. The imaging system for a cutaneous pigment disorder as claimed in claim 1, wherein the protective structure has dimensions compatible with a portable acquisition device (A).

3. The imaging system for a cutaneous pigment disorder as claimed in claim 1, wherein the acquisition device (A) comprises a multicellular structure, each cell containing a receiver.

4. The imaging system for a cutaneous pigment disorder as claimed in claim 3, wherein each cell comprises an emitter-receiver.

5. The imaging system for a cutaneous pigment disorder as claimed in claim 4, wherein for each image capture, the emitting angle and the receiving angle are oriented and equal with respect to the normal to the plane containing the skin.

6. The imaging system for a cutaneous pigment disorder as claimed in claim 3, wherein the acquisition device comprises a light source emitting in the visible band and means for acquiring the images (S1) simultaneously via the receivers.

7. The imaging system for a cutaneous pigment disorder as claimed in claim 1, wherein the positioning means comprise a rail on which one or more receivers are positioned and means for rotating the rail.

8. The imaging system for a cutaneous pigment disorder as claimed in claim 7, wherein the acquisition device comprises a single receiver or a single emitter-receiver on the rail and in that the rail is a sliding rail.

9. The imaging system for a cutaneous pigment disorder as claimed in claim 1, wherein the light source is multiwavelength in the visible and near-infrared bands, and/or the band of the first therapeutic window and/or the band of the second therapeutic window and/or the SWIR band, the one or more receivers corresponding to said wavelengths and being synchronized with the emitters, and in that the acquisition device comprises means for acquiring the images (S1) successively via the receivers at a rate of at least one image per wavelength and per viewing angle.

10. The imaging system for a cutaneous pigment disorder as claimed in claim 1, wherein the positioning window is arranged at the apex of the protective structure (1).

11. The imaging system for a cutaneous pigment disorder as claimed in claim 1, wherein the positioning window features a magnifying glass.

12. The imaging system for a cutaneous pigment disorder as claimed in claim 1, wherein it is portable.

13. The imaging system for a cutaneous pigment disorder as claimed in claim 1, wherein the acquisition device (A) is integrated into a mobile telephone.
